(19) United States
(12) Patent Application Publication
(10) Pub. No.: US 2005/0203105 A1
(43) Pub. Date: Sep. 15, 2005

(54) Composition and method for controlling alcohol-induced facial flushing in susceptible humans

(76) Inventors: Oon T. Tan, Boston, MA (US); Timothy J. Stafford, Boston, MA (US); Louis P. Scafuri, Cohasset, MA (US)

Correspondence Address: David Prashker;DAVID PRASHKER, P.C., P.O. Box 5387, Magnolia, MA, 01930, US

(21) Appl. No.: 11/009,559

(22) Filed: Dec. 10, 2004

Related U.S. Application Data
(63) Continuation of application No. PCT/US08/12601, filed on Apr. 23, 2003.

(51) Int. Cl. A61K 31/495; A61K 31/473; A61K 31/445

(52) U.S. Cl. 514/255.04; 514/317; 514/290

(57) ABSTRACT

The invention is a treatment method for controlling alcohol-induced flushing of the face and torso in susceptible humans. The method is a treatment for the person prior to his imbibing alcohol; employs a nonsedating H1-receptor histamine antagonist and a H2-receptor histamine antagonist in combination as orally administered medicaments; and is effective to block and avoid the flushing reaction in the susceptible person for a duration of about 3-4 hours' time.

### PRIORITY FILING

The present invention was first filed on Aug. 15, 2002 as U.S. Provisional Patent Application Ser. No. 60/403,687; and is presently pending as PCT International Patent Application No. PCT/US2003/012601 filed 23 Apr. 2003.

### FIELD OF THE INVENTION

The present invention is concerned generally with controlling the physiological effects of ethanol; and is directed particularly to compositions and a treatment method for controlling alcohol-induced facial flushing in susceptible humans.

### BACKGROUND OF THE INVENTION

Flushing is a transient reddening of the face, limbs, and areas of the torso, including the neck, upper chest and epigastric areas. The human flushing reaction is thus a physiological response of transient vasodilation; and flushing as a phenomenon is known to be mediated by a variety of different initiators, mechanisms of action, and underlying causes [J. K. Wilkin, Ann. Int. Med. 95: 468-476 (1981); Wilkin, J. K. and C. B. Rountree, Arch. Dermatol. 118: 109-111 (1982)].

Among the medically recognized forms of flushing are the following:

(i) flushing associated with Rosacea (a cosmetic disorder of the face characterized by telangeictasia, papules, pustules and eventually connective tissue hypertrophy);

(iii) flushing associated with eating (including hot beverages, auriculotem flushing, gustatory flushing, and dumping syndrome);

(iv) neurologic flushing (caused by anxiety, brain tumors, spinal cord lesions, orthostatic hypotension, migrane headaches, and Parkinson's disease);

(v) drug caused flushing (caused by vasodilators, calcium channel blockers, nicotinic acid, morphine, amyl nitrate and butyl nitrate, cholinergic agents, bromocriptine, thyroid releasing hormone, tamoxifen, cyproterone acetate, systemic steroids, and cyclosporin);

(vi) menopausal flushing (a symptom and side effect of menopause onset).

All of these reactions are recognizably separate and distinct conditions unrelated to each other—despite the flushing reaction commonly resulting as a consequence.

In addition to all of the above-identified maladies, there is another well known and medically documented form of facial and body flushing which is the direct result of drinking alcohol and is uniquely an alcohol-induced reaction in humans susceptible to this affliction. This alcohol-induced flushing reaction has particular social consequences and is described in specific detail hereinafter.

### A. Alcohol-Induced Flushing in Susceptible Humans

The biological cause of the human flushing response to alcohol consumption, as well as ethnic group differences to alcohol-induced flushing, has been and remains today an area of continuing interest, particularly among those involved with the study of alcohol use and alcoholism. Flushing of the face, limbs, and torso after drinking alcohol typically occurs in Oriental populations; and is marked by a distinctive facial reddening, an accelerated heartbeat, a blood pressure drop due to peripheral vasodilation, and other circulatory system changes, as well as by the presence of acetaldehyde and other alcohol metabolic abnormalities in the bloodstream.

Historical Developments:

The first researcher to study alcohol-induced flushing and to investigate racial ethnic differences in flushing was Wolff [P. Wolff, Science 175: 449-450 (1972)]. Using alcohol challenge tests, Wolff assessed flushing through photometric measures of skin reflectance; and found that adult persons of Mongoloid ancestry generally flushed, while persons of Caucasoid ancestry generally did not flush following the administration of a comparatively small amount of alcohol. Wolff also reported a second study of Mongoloid and Caucasoid infants who were given small doses of alcohol [P. Wolff, Am. J. Human Genetics 25: 193-199 (1973)]. These infants, each without any previous exposure to alcohol, showed the same kind of response differences for alcohol-induced flushing across racial ethnic groups as occurred for the adult subjects. These flushing differences between the racial groups did not appear to be the result of the development of tolerance through alcohol habituation.

Wolff himself suggested that facial flushing (while in itself a social embarrassment) also provides visible evidence of alcohol use; and that the flushing reaction is accompanied by other unpleasant symptoms that might have the effect of causing a reduction of alcohol use and abuse in those subpopulations who are susceptible to flushing. Wolff also found that susceptible persons of mixed Asian-European ancestry were similar to susceptible persons of Asian ancestry in their flushing response. These observations suggested that an individual's flushing reaction probably was inherited as autosomal dominant gene.

In addition, Wolff took note of the fact that there are subpopulations of Mongoloid ancestry (such as American Indians, Aleuts and Inuit Eskimos) who also flush with considerable frequency, but are not abstemious. Wolff suggested that the flushing response might lead to a reduction in alcohol use, but only in those subpopulations having relatively intact native cultures; and more recently acquired data support this interpretation. Hence, there is an absence of any “immunizing” effect among American Indians—despite the fact that they, like persons of Asian ancestry, are likely to show flushing following alcohol ingestion. Wolff's findings enticed many researchers to become interested in the human flushing response; and, in the due course of time, the metabolic bases of Mongoloid-Caucasoid differences soon came under scientific investigation.

More recent research studies have since revealed that two major enzyme systems are primarily involved with alcohol metabolism in-vivo. These appear to be the acetyl dehydrogenase (ADH) system and the acetaldehyde dehydrogenose (ALDH) system. Both of these enzyme systems exhibit genetic polymorphisms. The distinct variances in rate of reaction among isozymes that are related to and are reflected by ethnic group differences in response to alcohol use suggest a genetic basis for the observed individual and group differences in alcohol metabolism that, in turn, influence a person's alcohol consumption. There is a vast literature on this topic, much of which has to do with comparisons of Mongoloid-Caucasoid ADH and ALDH metabolism; and a portion of this literature is directed to the association of ADH and ALDH metabolism with facial flushing as well as with other alcohol-related symptoms. Published reviews of this body of literature are provided by A. W. Chan, Alcohol and Alcoholism 21: 93-104 (1986); Deitrich, R. A. and K. Spuhler, in Research Advances In Alcohol And Drug Problems, Vol. 8, Plenum Publishers, 1984; and Agarwal et al., Alcoholism: Clinical and Experimental Research 5: 12-16 (1981).

It is noted that researchers have reported substantial differences in ADH and ALDH enzymes between Mongoloids and Caucasoids, although there is considerable variation in this deficiency among the different Mongoloid groups [Goedde et al., Alcohol 2: 383-340 (1985)]. About 50 percent of Mongoloids appear not to have the ALDH-I isozyme. This deficiency results in impaired acetaldehyde oxidation leading to facial flushing and other cardiovascular symptoms. A more recent report [Miller et al., J. Studies Alcohol 49: 16-20 (1988)] suggests that the flushing response is, in fact, due to a rapid histamine response that may or may not be related to the ALDH-I deficiency. Also, studies of families from Hawaii, Taiwan, and Korea have indicated that there is considerable familial transmission of flushing after alcohol use; and concludes that such familial transmission is genetically based [See for example: Nagoshi et al., J. Studies Alcohol 49: 261-267 (1988); Johnson et al., Behavior Genetics 46: 171-178 (1984); Park et al., J. Studies Alcohol 45: 481-485 (1984)].

Fast and Slow Flushing Reactions:

For definitional purposes, an alcoholic drink will typically contain about one (1) fluid ounce of 80-100 proof (40-50% ethanol), with or without another liquid being present to form an alcoholic beverage. Accordingly, with regard to the alcohol-induced flushing reaction itself, a major difference and distinction is said to exist between persons who are “fast flushers” (i.e., those who flushed after consuming one alcoholic drink or less) and persons who are “slow flushers” (i.e., those who must imbibe more than one alcohol drink to produce the flushing response). The distinction between fast and slow flushing reactions in humans is not arbitrary. To the contrary, Wolff's pioneer studies (published in 1972 and 1973) assessed the initiation of the flushing reaction as occurring either (a) for some persons after consuming only one alcoholic drink; or (b) for other individuals only after imbibing multiple alcoholic drinks. Subsequently published reports confirmed the existence of the marked differences between “fast” and “slow” flushing reactions. [See for example: Schwitters et al., J. Studies Alcohol 42: 1259-1262 (1982); Park et al., J. Studies Alcohol 45: 481-485 (1984)].

Distinct Racial Differences:

The association between the flushing response and the imbibing of alcohol is particularly prevalent among persons of Asian ancestry and is especially marked among individuals of Japanese descent [Nakawatase et al., J. Stud. Alcohol 54: 48-53 (1993) and the references internally cited therein]. The result of much empirical research appears to indicate that individuals of Asian ancestry are particularly more alcohol sensitive; and thus are more likely to respond to drinking alcohol with a marked facial and torso flushing in comparison to persons of European ethnicity [Nagoshi et al., J. Stud. Alcohol 49: 261-267 (1988); Johnson et al., Conference on Epidemiology Of Alcohol Use And Abuse Among US Minorities, Bethesda, Md., Sep. 11-14, 1985; Suwaki, A. and H. Ohara, J. Stud. Alcohol 46: 196-198 (1984)].

### B. Proposed Treatments for the Alcohol-Induced Flushing Reaction

A number of different agents and treatment approaches to mediate or avoid the onset of alcohol-induced flushing in humans, particularly in Oriental subpopulations, have been reported in the scientific literature. The reported attempts appear to have had varying degrees of success and presently include the following;
1. Aspirin attenuation of alcohol-induced flushing
2. Naloxone blockade of alcohol-induced flushing
3. Clonidine blockade of alcohol-induced flushing in a patient with carcinoid syndrome
4. Histamine receptor blockade of alcohol-induced flushing.
   Aspirin Attenuation of Alcohol-Induced Flushing:

Perhaps the best example of aspirin attenuation of alcohol-induced flushing has been reported by Truitt et al. [Alcohol; Suppl. 1: 595-599 (1987)]. Aspirin (acetylsalicylic acid or “ASA”) was tested in a group of 8 Oriental and 3 Occidental subjects who were shown in a previous study to respond to small doses of ethanol (0.06-0.25 g/kg) with facial flushing. These persons were compared to a group of 11 non-flushing Occidental subjects—using a larger ethanol dose (0.37 g/kg) to determine if similar effects could be produced in less sensitive individuals. Control tests of blood ethanol and acetaldehyde (AcH) levels, facial and neck skin temperatures, body sway (Romberg test), blood pressure, heart rate and Subjective High Assessment Scales (SHAS) were conducted before and at 15, 30, 60 and 90 minutes' time after drinking ethanol (as vodka in orange juice). The tests were repeated one week later one hour after receiving 0.64 gm of aspirin orally.

The data revealed that aspirin produced slight changes in the early absorption of ethanol and small decreases in AcH levels in the flushing and non-flushing groups. Facial flushing was markedly reduced in the flushing group, but was slightly increased in the non-flushing Occidentals. Body sway was reduced by aspirin in both groups of subjects. An alcohol induced increase in heart rate in the flushing group of subjects was reduced by aspirin with no change in blood pressure. The SHAS subjective parameters were widely variable but indicated that aspirin produced reduced sleepiness and earlier relaxation in the flushing group. It was concluded that aspirin can block alcohol-induced facial flushing in sensitive subjects and alter some subjective feelings of alcohol intoxication.

Naloxone Blockade of Alcohol-Induced Flushing:

Naloxone is an opiate antagonist typically used in rehabilitation of opiate addicts. Its pharmacology in humans is well established [Jasinki et al., J. Pharmacol. Exp. Ther. 157: 420 (1967)]; and its method of preparation is publicly available [U.S. Pat. No. 3,254,088 (1966)].

Naloxone has been employed to block alcohol-induced rosacea flushing as reported by J. E. Bernstein and K. Soltani [Brit. J. Dermatol. 107: 59-62 (1982)]. As stated therein, the roles of endogenous opioid peptides and histamine were evaluated in the pathophysiology of alcohol-induced facial flushing in rosacea. Non-diabetic patients with rosacea ingested 360 ml of 6% ethanol after receiving either subcutaneous naloxone hydrochloride or oral chlorpropamide maleate. Only pretreatment with naloxone blocked the alcohol-induced rosacea flushing (AIRF), suggesting an active role of endogenous enkephalin and/or endorphin in this vascular reactivity.

A second example of naloxone blockade of alcohol-induced flushing in humans is provided by Baraniuk et al. [Alcohol Clin. Exp. Res. 11: 518-520 (1987)]. As reported therein, the effects of imbibing ethanol and the subsequent administration of intravenous naloxone were studied in double-blind, placebo-controlled fashion using a group of six male chlorpropamide alcohol flushers (CPAF) and a group of 13 non-flushing males. The effects of ethanol intoxication upon fine motor control were also measured by a typing test. When sober, the two groups performed in comparable fashion. When intoxicated, the CPAF group displayed significantly greater impairment than the non-flushing group as measured by typing errors committed in 3 minutes' time. Chlorpropamide alcohol flushers also appeared to be more sensitive to ethanol. When administered, naloxone reversed this effect for individuals in the CPAF group; but naloxone had no effect in the non-flushing group. It was concluded, therefore, that unlike the normal non-flushing group, the CPAF group demonstrated an increased sensitivity to ethanol that was partially antagonized by naloxone.

Clonidine Blockade of Alcohol-Induced Flushing:

The most pertinent report in the scientific literature regarding the use of clonidine, an α-adrenengic agonist, and the alcohol-induced flushing reaction appeared in 1982 [J. K. Wilkin and C. B. Rountree, Arch. Dermatol. 118: 109-111 (1982)]. This report concerned a 29-year old woman who had a nine-year history of excessive flushing reactions to different beverages. Gustatory agents that provoked her flushing included hot beverages, alcohol, and chocolate. Also, four years earlier, a diagnosis of carcinoid syndrome had been made for her on the basis of clinical findings, excessive urinary 5-hydroxyinodole acetic acid excretion, and a liver biopsy specimen that disclosed a metastatic carcinoid tumor. The only medication she had regularly taken during the past four years had been 25 mg of oral chlorpromazine hydrochloride every night; but her use of this medication was discontinued several months before the experimental testing began.

The following challenging agents were studied using this woman as a test subject: 180 mL of water at 60° C.; 180 mL of ethyl alcohol-fortified red wine (20% ethyl alcohol); and 30 g of milk chocolate. All flushing studies were conducted between 7 AM and 10 AM after an overnight fast (water only) and the administration of pharmacologic agents. Challenges with provocative agents were conducted at intervals of 24 hours or more to avoid a possible refractory period. After a baseline malar temperature was achieved, the subject was given the provocative agent by mouth. After “control” flushing studies, consisting of one challenge for each provocative agent, the patient was rechallenged after prior treatment with several oral medications. Each medication regimen was administered for two weeks. Rechallenges with water at 60° C., red wine, and milk chocolate were evaluated during the second week of each treatment course. Following this procedure, all rechallenges occurred during the ninth through the 14th day of pharmacologic treatment.

The first test regimen consisted of 0.05 mg of oral clonidine hydrochloride twice a day for two weeks. The second test regimen consisted of 4 mg of oral chlorpheniramine maleate four times a day. During the third two-week test period, 300 mg of oral cimetidine was given four times a day. Finally, during the last two-week test period, a combination of 4 mg of oral chlorpheniramine maleate and 300 mg of oral cimetidine was given four times a day.

The results of 14 challenges were examined and evaluated (including the results of 11 challenges with prior pharmacologic treatment and three without). In the absence of prior pharmacologic treatment, the woman had moderate flushing reaction to both red wine and hot water and a strong flushing reaction to chocolate. Prior treatment with chlorpheniramine did not affect the flushing response. Also, clonidine, cimetidine, and the combination of chlorpheniramine and cimetidine blocked the flushing responses to red wine and chocolate, but not to hot water.

Histamine Receptor Blockade of Alcohol-Induced Flushing:

Within this category, a major portion of the investigations reported in the scientific literature comprise only scattered bits of information which are tenuously related. These publications employed a variety of different agents, as described below.

H2-Receptor Histamine Antagonists

Exemplifying such publications are those reports concerned with the effect of Histamine-2 receptor antagonists on blood alcohol levels [See for example: Weinberg et al., J. Gen. Intern. Med. 13: 594-599 (1998)]. The reported data and conclusions showed that cimetidine and ranitidine—but not other H2 histamine antagonists—can cause small elevations of serum alcohol levels when alcohol and the H2 blocker are administered concurrently. In the stated view, the effect of administering any H2 histamine antagonist on blood alcohol is unlikely to be clinically relevant.

Another published example reporting the effect of cimetidine on ethanol concentrations in fasting men and women is provided by Clemmesen et al. [Scand. J. Gastroenterol. 32: 217-220 (1997)]. Their reported data and conclusions show that: (a) the ethanol elimination rate was unchanged by the administration of cimetidine to the subject; and (b) that cimetidine does not influence the ethanol concentration-time curve when ethanol is ingested on an empty stomach.

Other publications and reported scientific investigations reveal substantially the same information: The commonly available H2-receptor histamine antagonists can cause small increases in blood alcohol concentrations, but the absolute increase is very small [A. G. Fraser, Drug Metabol. Drug Interact. 14: 123-145 (1998)]. Also, individuals who were administered ethanol orally before and after treatment with cimetidine revealed higher blood ethanol levels after cimetidine administration [Kawashima et al., Alcohol Clin. Exp. Res. 20 (Suppl. 1): 36A-39A (1996)]. Moreover, under conditions mimicking human social drinking habits, ranitidine increases blood alcohol to levels which impair psychomotor skills needed for driving a car [Arora et al., Am. J. Gastroenterol. 95: 208-213 (2000)]. In addition, when low doses of alcohol (below 0.3 g/kg) are given by mouth, the administration of H2 receptor antagonists results in an increase in the blood ethanol concentrations [Nemesanszky, E. and A. Csepregi, Onv. Hetil. 137: 1309-1313 (1996)]. Finally, the evident enhancement of alcohol-induced hypoglycemia caused by H2 receptor antagonists (such as cimetidine, ranitidine and famotidine) is not dependent upon the increase of ethanol absorption from the gastrointestinal tract, but represents a specific effect of H2 blockers on glucose metabolism [Czyzyk et al., Arzneimittelforschung 47: 746-749 (1997)].

H1-Receptor Histamine Antagonists

An extensive body of published research exists concerning central nervous system (CNS) effects of H1-antagonists. There is great interest in this area due to the well-known adverse CNS effects associated with first-generation H1 antagonists, particularly in comparison to the new second-generation agents having nonsedative properties. Because the CNS effects of H1 antagonists are complex and cannot be reflected in one clinical measurement, a variety of assessments evaluating CNS function are required. These assessments range from the subjective (e.g., self-rating of drowsiness) to the objective (e.g., 24 h EEG sleep latency), and from the simple (e.g., critical flicker fusion) to the complex (e.g., actual driving). When these tests are applied to the evaluation of the currently available H1-receptor histamine antagonists, it is clear that there are major differences and marked distinctions between the older first-generation H1 antagonists and the newer, nonsedating, second-generation compositions.

Much of the recently published scientific literature regarding H1 blockers is concerned with measuring the substantive differences between first generation and second generation H1-receptor histamine antagonists. Merely exemplifying such investigations is the published work of Welch et al. [Clin. Allergy Immunol. 17: 337-388 (2002)]. As reported therein, when used at the recommended dosages, all the second-generation H1-antagonists are clearly less sedating than their predecessors. These newer second-generation medications do not cross the blood-brain barrier readily; are highly specific for H1-receptors; have little to no anticholinergic, antiserotoninergic, or anti-alpha-adrenergic effects; and do not enhance the adverse CNS effects of alcohol or other CNS-active substances such as the benzodiazepines. In addition, since most second-generation H1-antagonists are relatively nonsedating, their usuage and benefit/risk ratios will be determined more by their concomitant and incidental properties—such as not causing cardiovascular system (CVS) adverse effects (e.g., potential to cause cardiac arrhythmias); their potency; their time for onset and duration of action; their ease of administration; and their cost.

Use of Multiple Histamine Antagonists for Preventing Alcohol-Induced Flushing

Two publications in the scientific literature report attempts to block alcohol-induced flushing using a combination of H1-receptor and H2-receptor histamine antagonists. Both of these published reports reveal some of the major obstacles in developing a clinically acceptable and medically efficacious treatment to prevent the alcohol-induced flushing reaction in a susceptible person.

The earlier report is a Letter To The Editor which appeared in the Aug. 18, 1979 issue of The Lancet [Tan et al., Lancet ii: 365 (1979)]. The published letter briefly described using the measurement of tissue oxygen concentration in the skin via electrodes to assess changes in superficial skin blood flow in response to peripheral vasodilators. As reported therein, susceptible human subjects were given 30-50 ml of sherry to induce facial flushing; and attempts to block the forthcoming flushing reaction were made by orally giving chlorpheniramine (a first generation H1 histamine antagonist) and 200 mg of cimetidine (a H2 histamine antagonist) alone and then in combination to the subjects 30 minutes before the challenge with sherry. It was observed that, while the chlorpheniramine given alone did not affect the rise in skin oxygen tension in the subjects, the cimetidine given alone did noticeably reduce the increase in oxygen concentration. When given in combination, however, the two antagonists abolished the rise in oxygen tension. In this manner, the flushing reaction in the subjects was prevented by the first generation H1 antagonist and the H2 antagonist in combination.

The latter publication [Tan et al., Brit. J. Dermatol. 107: 647-652 (1982)] is a followup to their 1979 Letter To The Editor; and reports an expanded study of the effects of chlorpheniramine and cimetidine in combination for suppression of alcohol-induced flushing This study employed the same test parameters as in the earlier 1979 report; but now included measurements of blood alcohol levels as well as the side effect consequences for the treatment regimen. The reported results showed: (a) that the suppression of alcohol-induced flushing is due to a lowering of alcohol blood levels in the susceptible subjects; (b) that the suppression effect of the chlorpheniramine and cimetidine in combination could be overcome by increasing the amount of alcohol ingested by the subject; and (c) that another effect of giving chlorpheniramine and cimetidine in combination is upon gastric motility, with a concomitant reduction in the rate of absorption of alcohol from the gastrointestinal tract. Equally important and of particular note is the explicit recognition that the administered chlorpheniramine not only increases the rate of absorption of alcohol in the susceptible subject, but also potentiates the sedative action of the alcohol absorbed in this circumstance—such that drowsiness can occur in persons not otherwise subject to it. Thus, a sedative effect and drowsiness is a direct outcome and concomitant consequence of the antihistamines employed in combination to suppress the alcohol-induced flushing in the susceptible subject.

### SUMMARY OF THE INVENTION

The present invention provides a method for controlling alcohol-induced flushing in a susceptible human, said method comprising the steps of:

administering to the susceptible human an effective amount of at least one nonsedating H1-receptor histamine antagonist;

concurrently administering to the susceptible human an effective amount of at least one H2-receptor histamine antagonist; and

waiting a predetermined time period after said nonsedating H1-receptor histamine antagonist and said H2-receptor histamine antagonist are administered before the susceptible human imbibes alcohol.

### DETAILED DESCRIPTION OF THE INVENTION

The present treatment method employs two different medicaments to control and prevent the onset of alcohol-induced flushing of the face and torso in susceptible humans. The treatment uses at least one nonsedating H1 antagonist in combination with at least one H2 antagonist, this combination of medicaments being preferably orally administered prior to the event or circumstance where a consumption of alcoholic beverages is expected to occur. The concurrent administration of these nonsedating H1 and H2 antagonist medications will serve to control flushing for a limited duration of hours; and markedly reduce or completely eliminate the facial and torso flushing which accompanies the drinking of alcohol for certain susceptible individuals.

In the preferred mode of treatment, the combination of a nonsedating H1-receptor histamine antagonist compound and a H2-receptor histamine antagonist compound are orally administered concurrently about 30-45 minutes before beginning to drink alcohol. The duration of control and the prevention/reduction of the flushing reaction typically last about three to four hours in duration.

Over this effective treatment time period (about 3-4 hours), the administered combination of antagonists will block multiple histamine receptor sites within the susceptible human; control and prevent flushing of the face and torso for the individual; and concomitantly avoid causing two major undesired side effects—sedation and drowsiness—particularly in persons often subject to these incidental problems.

In view of the foregoing, the disclosure will provide detailed information concerning: the range and variety of medicaments (the nonsedating H1-receptor histamine antagonists and H2-receptor histamine antagonists) presently available for use in the treatment method; the operable and preferred dosages, administrations, frequency of use, and precautions for each of the medicaments employed; and individual case histories of some susceptible humans who utilized the present treatment method to control and avoid the onset of facial and torso flushing caused by imbibing alcohol on social occasions.

### I. Antihistamines

Antihistamines comprise a broad class of pharmacologic agents that act at three different histamine receptor sites: The H1-receptor antagonists including the first-generation, centrally acting, H1-receptor antagonists (e.g., diphenhydramine) and the newer, second-generation, nonsedating H1 blockers (e.g., loratadine); the H2-receptor antagonists, such as cimetidine, which work primarily at H2 receptor sites and cause an inhibition of gastric secretion; and the H3-receptor antagonists which are still experimental antihistamines which act specifically at H3 receptor sites. The H1, H2, and H3 receptors constitute the three different kinds of histamine receptors that have been pharmacologically identified to date.

The importance of proper antihistamine identification (aside from receptor site differences) has increased with the recognition of potentially life-threatening cardiac toxicity from relatively small exposures to terfenadine. Also patients who ingest the newer nonsedating antihistamines have fewer central anticholinergic symptoms than those who ingest any of the first-generation agents. Classification of each type of antihistamine thus proceeds on the basis of either specific physiologic effect (e.g., sedating vs. nonsedating) or on the basis of chemical structure (e.g., alkylamine vs. piperidine derivatives).

The clinical indications and uses of antihistamines are well recognized and established:
(i) All antihistamines are well absorbed following oral administration;
(ii) Most achieve peak plasma concentrations within 3 hours with the onset of symptoms occurring between 30 minutes and 2 hours of ingestion;
(iii) The duration of action ranges from 3 hours to more than 24 hours;
(iv) Hepatic metabolism is the primary route of elimination for antihistamines.

### A. The H1-Receptor Histamine Antagonists

All of the conventionally available H1-receptor histamine antagonists are reversible, competitive inhibitors of the actions of histamine. The structure of almost all of the “classic” H1 antihistamines have a tertiary amino group linked by two- or three-atom chain to two aromatic substituents and conform to the general formula shown below, where Ar is aryl and X is a nitrogen or a carbon atom or a C—O-ether linkage.

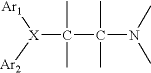

H1-receptor blocking drugs have an established and valued place in the symptomatic treatment of various immediate hypersensitivity reactions, in which their usefulness is attributable to their antagonism of endogenously released histamine (one of several autoacids that elicit an allergic response). In addition, the central nervous system (CNS) properties for some of the H1 antagonist series are of considerable therapeutic value in suppressing motion sickness.

H1 Antihistamine Actions Generally:

### Pharmacology

H1-antihistamines competitively antagonize histamine at the H1 receptor site, but do not bind with histamine to inactivate it. Terfenadine and astemizol, the most specific H1-antagonists available, bind preferentially to peripheral rather than central H1 receptors. However, H1-antihistamines do not block histamine release, or antibody production, or antibody interactions. Rather, they antagonize in varying degrees most of the pharmacological effects of histamine.

Some H1-antagonists also have anticholinergic (drying), antipruritic and sedative effects. For example, H1-antihistamines with predominant sedative effects are used as nonprescription sleeping aids. Nevertheless others, such as terfenadine and astemizole, have little or no anticholinergic or sedative effects at all. In addition, others such as cyproheptadine and azatadine have recognized antiserotonin activity; while other H1-antihistamines with antiemetic effects are useful in the management of nausea, vomiting and motion sickness. Conversely, gastro-intestinal upset is a frequent side effect of those H1-antihistamines which chemically are ethylenedlamines.

### Pharmacokinetics

With a few exceptions, H1-antagonists are well absorbed following oral administration; have an onset of action within 15 to 30 minutes; are maximally effective within 1 to 2 hours; and have a duration of about 4 to 6 hours, although some of the drugs are much longer acting. Most are metabolized by the liver. H1-antihistamine metabolites and small amounts of unchanged drug are excreted in the urine. Small amounts may be excreted in breast milk.

For example, terfenadine's effects begin in 1 to 2 hours, reach a maximum in 3 to 4 hours and last in excess of 12 hours. Terfenadine reaches peak plasma levels in 2 hours and has an elimination half-life of 20 hours. It is 97% protein bound. Fecal excretion accounts for 60% the dose, with 40% eliminated via the urine. Almost all of the dose is eliminated as metabolites.

In comparison, astemizole has a slow onset of action and its effects last up to 24 hours, based on once-a-day dosing. It is rapidly absorbed and reaches peak plasma concentrations within 1 hour; its absorption is reduced by 60% when taken with food. Its half-life is biphasic: 20 hours for the distribution phase and 7 to 11 days for the elimination phase. The drug is 96.7% protein bound. Approximately 40% to 50% of the astemizole dose is excreted in the urine by 4 days, with 50% to 70% eliminated via the feces by 14 days. All of the dose is eliminated as metabolite. The principal metabolite may have some antihistaminic activity.

### Contraindications

Hypersensitivity to antihistamines can occur in newborn or premature infants, nursing mothers, narrow-angle glaucoma; stenosing peptic ulcer; symptomatic prostatic hypertrophy; asthmatic attack; bladder neck obstruction, pyloroduodenal obstruction; and monoamine oxidase inhibitor (MAOI) therapy.

Phenothiazine antihistamines such as trimeprazine, promethazine and methdilazine should not be used with comatose patients; CNS depression from barbiturates, general anesthetics, tranquilizers. alcohol, narcotics or narcotic analgesics; previous phenothiazine idiosyncrasy, jaundice or bone marrow depression; and acutely ill or dehydrated children because there is greater susceptibility to dystonias.

Similarly, astemizole and terfenadine can cause QT interval prolongation/ventricular arrhythmias. Rare cases of serious cardiovascular adverse events, including death, cardiac arrest, torsade de pointes and other ventricular arrhythmias, have been observed in the following clinical settings frequently in association with increased terfenadine and astemizole (including metabolite) levels which lead to electrocardiographic QT prolongation:
i. Overdose including single terfenadine doses as low as 360 mg and astemizole doses as low as 20 to 30 mg/day.
ii. Significant hepatic dysfunction.
iii. Concomitant administration of erythromycin, ketoconazole or itraconazole.

Terfenadine and astemizole are also contraindicated in patients taking ketoconazole, itraconazone or erythromycin and in patients with significant hepatic dysfunction. The most commonly described drug interactions have involved a combination of terfenadine with erythromycin. Similar reactions have been described with both terfenadine and astemizole in combination with other macrolide antibiotics (with the exception of azithromycin), azole antifungal agents, cisapride, cimetidine, fluexetine, nefazodone, omeprazole, protease inhibitors (e.g., nelfinavir, indinavir, ritonavir), and even grapefruit juice.

Prolonged QT syndrome and cardiac arrhythmias rarely have been described with loratadine. An exemplary listing of some drug interactions is presented by Table 1 below.

TABLE 1
Drug Interactions*
Antihistamine Drug Interactions

| Precipitant | | | |
|---|---|---|---|
| drug | Object drug | | Description |
| Azole | Antihistamines- | ↑ | Astemizole and terfenadine |
| Antifungals | Astemizole | | plasma levels (including metabolite |
| Fluconazole | Terfenadine | | levels) may be increased, which |
| Itraconazole | | | may lead to serious cardiovascular |
| Ketoconazole | | | effects (see Warning Box). |
| Miconazole | | | |
| Macrolide | Antihistamines- | ↑ | Astemizole and terfenadine |
| antibiotics | Astemizole | | plasma levels (including metabolite |
| | Terfenadine | | levels) may be increased, which |
| | | | may lead to serious cardiovascular |
| | | | effects (see Warning Box). |
| MAO | Antihistamines | ↑ | MOAIs may prolong and |
| inhibitors | | | intensify the anticholinergic |
| Antihistamines | MAO | ↑ | effects of the antihistamines. |
| | inhibitors | | Use with phenothiazine |
| | | | antihistamines may cause |
| | | | hypotension and extrapyramidal |
| | | | reactions. Dexchlorpheniramine |
| | | | may cause severe hypotension |
| | | | when given with an MAOI. |
| Antihistamines | Alcohol, CNS | ↑ | Additive CNS depressant effects |
| | depressants | | may occur. This may be less likely |
| | | | with astemizole, loratadine and |
| | | | terfenadine. |

↑ = Object drug increased
*Reproduced from: Drug Facts and Comparisons, 1995, p. 1047.

Differences between the First Generation and the Second Generation of H1-Receptor Histamine Antagonists:

All H1 histamine antagonists are reversible competitive inhibitors of histamine receptors, but there are marked differences between the first generation and the second generation agents. First-generation H1-receptor blockers are potent competitive inhibitors of muscarinic receptors and may cause anticholinergic syndrome (e.g., sinus tachycardia, dry skin, dry mucous membranes, dilated pupils, ileus, urinary retention, agitated delirium). In addition, first generation H1-antihistamines disrupt cortical neurotransmission and block fast sodium channels. These effects exacerbate sedation and seizure activity; and may cause cardiac conduction delays manifested by widening of the QRS interval. The phenothiazine class of H1-antihistamines (e.g., promethazine) also has alpha-adrenergic blocking activity and may cause hypotension.

In comparison, the second generation of H1-receptor blockers are peripherally selective antagonists and are nonsedating agents. The second generation of H1-receptor antagonists also have a prolonged duration of action and offer a low incidence of drowsiness.

Six different chemical classes of H1-antihistamines are known and typically are set forth in chemical structure as follows^{#:}
[^{#} Some authorities have placed these structural classes in alternative category and chemical class schemes. See, for example, Table 2 herein.]

1. Alkylamines (e.g., brompheniramine, triprolidine);

2. Ethanolamines (e.g., clemastine, diphehydramine, doxylamine);

3. Ehtylenediamines (e.g., tripelennamine);

4. Phenothiazines (e.g., promethazine);

5. Piperidines derivatives (e.g., astemizole, fexofenadine, loratadine, and terfenadine);

6. Piperazines (e.g., cetirizine, meclizine).

TABLE 2
Pharmacology^{t}
Antihistamines: Dosages and Effects

| | | Dosing | | Antihistaminic | Anticholinergic | Antiemetic |
|---|---|---|---|---|---|---|
| Antihistamine | Dose¹ (mg) | interval² (hrs) | Sedative effects | activity | activity | effects |
| Ethanolamines | | | | | | |
| Carbinoxamine | 4 to 8 | 6 to 8 | ++ | + to ++ | +++ | ++ to +++ |
| Clemastine | 1 | 12 | ++ | + to ++ | +++ | ++ to +++ |
| Diphenhydramine | 25 to 50 | 6 to 8 | +++ | + to ++ | +++ | ++ to +++ |
| Ethylenediamines | | | | | | |
| Pyrilamine | 25 to 50 | 6 to 8 | + | + to ++ | ± | − |
| Tripelennamine | 25 to 50 | 4 to 6 | ++ | + to ++ | ± | − |
| Alkylamines | | | | | | |
| Brompheniramine | 4 | 4 to 6 | + | +++ | ++ | − |
| Chlorpheniramine | 4 | 4 to 6 | + | ++ | ++ | − |
| Dexchlorpheniramine | 2 | 4 to 6 | + | +++ | ++ | − |
| Triprolidine | 2.5 | 4 to 6 | + | ++ to +++ | ++ | − |
| Phenothiazines | | | | | | |
| Methdilazine | 8 | 6 to 12 | + | ++ to +++ | +++ | ++++ |
| Promethazine | 12.5 to 25 | 6 to 24 | +++ | +++ | +++ | ++++ |
| Trimeprazine | 2.5 | 6 | ++ | ++ to +++ | +++ | ++++ |
| Piperidines | | | | | | |
| Azatadine | 1 to 2 | 12 | ++ | ++ | ++ | − |
| Cyproheptadine | 4 | 8 | + | ++ | ++ | − |
| Phenindamine | 25 | 4 to 6 | −³ | ++ | ++ | − |
| Miscellaneous | | | | | | |
| Astemizole | 10 | 24 | ± | ++ to +++ | ± | − |
| Loratadine | 10 | 24 | ± | ++ to +++ | ± | − |
| Terfenadine | 60 | 12 | ± | ++ to +++ | ± | − |

+++++ = Very high,
+++ = high,
++ = moderate,
+ = low,
± = low to none.
¹usual single oral adult dose.
²for conventional dosage forms.
³Stimulation possible
^{t}Reproduced from: Drug Facts And Comparisons, 1995, p. 1043.

In addition, the following information is also noted for particular H1-receptor histamine antagonists:

(i) Alkylamine derivatives (e.g., chlorpheniramine, brompheniramine, triprolidine) are among the most potent H1-antihistamines. They induce more CNS stimulation and cause less drowsiness than other antihistamines.

(ii) Ethanolamine derivatives (e.g., doxylamine, diphehydramine, bromodiphenhydramine) have strong atropien-like activity; and drowsiness is common. Adverse gastrointestinal effects, however, are uncommon. Seizures and cardiac conduction delays are common especially in massive diphenhydramine ingestions.

(iii) Ethylenediamine derivatives (e.g., pyrilamine, tripelennamine, antazoline) have weak CNS effects. Adverse GI effects are common.

(iv) Phenothiazine derivatives (e.g., promethazine, trimeprazine, methdilazine) possess considerable anticholinergic activity and minimal GI adverse effects.

(v) Piperidine derivatives generally have a prolonged duration of action and a low incidence of drowsiness. Specific examples include hydroxyzine, cetirizine, and meclizine.

(vi) Piperidine derivatives (e.g., terfenadine, astemizole, and loratadine) are peripherally selective H1-antagonists with few GI adverse effects; and cause a low incidence of drowsiness as nonsedating antihistamines.

### Sedating First-Generation H1-Antihistamines

The classic or first-generation H1-antihistamine can cause delirium, sedation, and anticholinergic symptoms in any patient. Examplifying such sedating H1-antagonists are chlorpheniramine, hydroxyzine, and diphenhydramine.

### Nonsedating Second Generation H1-Antihistamines

The nonsedating second generation antihistamines differ markedly from their first generation predecessor antihistamines in that the second generation agents are primarily piperidenes derivatives; do not partitition into the CNS; and typically have long half-lives of activity. The half-life of loratadine, for example, is typically 10 hours, but may be more than doubled in half-life duration when used in excess dosages. Representing and examplifying the presently known, nonsedating second-generation H1-antagonists are loratadine, desloratadine (a breakdown product of loratadine), terfenadine, astemizole, and fenoxfenadine.

Among the preferred nonsedating H1-receptor antagonists are fenoxadine, loratadine, and desloratadine. These formulations are preferred primarily because they are the safest peripherally selective H1-receptor histamine antagonists available to date. Each of these preferred agents has a distinct physiological advantage because they bind selectively to peripheral H1 receptors and have a lower binding affinity for the cholinergic and alpha-adrenegic receptor sites than other H1-antihistamines. This group of second generation, nonsedating H1-antihistamines also eliminate and avoid many of the adverse effects commonly associated with H1-receptor binding—including central nervous system (CNS) depression, blurred vision, dry mouth, and tachycardia.

The nonsedating second generation of H1 antihistamines (especially loratadine and desloratadine) also are known to inhibit the potassium rectifier currents and, thus, slow repolarization. This is manifested clinically as prolongation of the QT interval and torsade de pointes. Some agents such as astemizole, however, have recently been removed from the pharmaceutical market. Also, terfenadine has been removed from the market and replaced by fexofenadine, which is a pharmacologically active metabolite of terfenadine. Fexofenadine has not been associated with torsade de pointes in volunteer and animal studies.

Accordingly, among these nonsedating, second generation of selective H1-antagonists, the most preferred pharmacological compound are loratadine and its active breakdown product, desloratadine. A summary description of these two most preferred agents is given below.

### Loratadine (CLARATIN)/Desloratadine (CLARINEX)

Loratadine is a white to off-white powder not soluble in water, but very soluble in acetone, alcohol, and chloroform. It has a molecular weight of 382.89, and empirical formula of C₂₂H₂₃CIN₂O₂; and its chemical name is ethyl 4-(8-chloro-5,6-dihydro-11H-benzo[5,6]cyclohepta[1, 2-b]pyridin-11-ylidene)-1-piperidinecarboxylate. An active metabolic breakdown product of loratadine is desloratadine, which is available under the brand name CLARINEX.

CLARITIN tablets contain 10 mg micronized loratadine, an antihistamine, to be administered orally. They also contain the following inactive ingredients; corn starch, lactose, and magnesium stearate. CLARINEX tablets contain 5 mg desloratadine, to be administered orally.

CLARITIN syrup contains 1 mg/mL micronized loratadine, an antihistamine, to be administered orally. It also contains the following inactive ingredients: citric acid, edetate disodium, artificial flavor, glycerin, propylene glycol, sodium benzoate, sugar, and water. The pH is between 2.5 and 3.1. In comparison, CLARITIN REDITABS (loratadine rapidly-disintegrating tablets) contain 10 mg micronized loratadine, an antihistamine, to be administered orally. It disintegrates in the mouth within seconds after placement on the tongue, allowing its contents to be subsequently swallowed with or without water. Loratadine rapidly-disintegrating tablets also contain the following inactive ingredients: citric acid, gelatin, mannitol, and mint flavor.

Clinical Pharmacology of Loratadine:

Loratadine is a long-acting tricyclic antihistamine with selective peripheral histamine H₁-receptor antagonistic activity. Loratadine's effects begin within 1 to 3 hours, reaching a maximum at 8 to 12 hours and last more than 24 hours. It is rapidly absorbed and extensively metabolized to an active metabolite, known as desloratadine (chemically, descarboethoxyloratadine). The mean elimination half-life is 84 hours for loratadine and 28 hours for the metabolite product. Approximately 80% of the loratadine dose is equally distributed between urine and feces in the form of metabolic products after 10 day. Loratadine is 0≈97% protein bound; in comparison, the metabolite is 73% to 77% protein bound.

Human histamine skin wheal studies following single and repeated 10 mg oral doses of loratadine have shown that the drug exhibits an antihistaminic effect beginning within 1 to 3 hours, reaching a maximum at 8 to 12 hours. and lasting in excess of 24 hours. There was no evidence of tolerance to this effect after 28 days of dosing with loratadine. Whole body autoradiographic studies in rats and monkeys, radiolabeled tissue distribution studies in mice and rats. and in vivo radioligand studies in mice have shown that neither loratadine nor its metabolites readily cross the blood-brain barrier. Radioligand binding studies with guinea pig pulmonary and brain H1-receptors indicate that there was preferential binding to peripheral versus central nervous system H1-receptors.

Pharmacokinetics of Loratadine:

Loratadine was rapidly absorbed following oral administration of 10 mg tablets, once daily for 10 days to healthy adult volunteers with times to maximum concentration (Tₘₐₓ) of 13 hours for loratadine and 2.5 hours for its major active-metabolite, descarboethoxyloratadine. Based on a cross-study comparison of single doses of loratadine syrup and tablets given 10 healthy adult volunteers, the plasma concentration profile of descarboethoxyloratadine for the two formulations is comparable. The pharmacokinetics of loratadine and descarboethoxyloratadine are independent of dose over the dose range of 10 to 40 mg and are not altered by the duration of treatment. In a single-dose study, food increased the systemic bioavailability (AUC) of loratadine and descarboethoxyloratadine by approximately 40% and 15%, respectively. The time to peak plasma concentration (Tₘₐₓ) of loratadine and descarboethoxyloratadine was delayed by 1 hour. Peak plasma concentrations (Cₘₐₓ) were not affected by food.

### B. H2-Receptor Histamine Antagonists

The H2 blockers are reversible, competitive antagonists of the actions of histamine on H2 receptors. They are highly selective in their action and are virtually without effect on H1 receptors. The most prominent of the effects of histamine that are mediated by H2 receptors is stimulation of gastric acid secretion; and it is the ability of the H2 blockers to inhibit this effect that explains much of their conventional medical importance. Despite the widespread distribution of H2 receptors in the body, H2 blockers interfere remarkably little with physiological function other than gastric secretion.

H2 receptors are thus primary regulators of gastric acid secretion. In the CNS, histamine (H1, H2) modulates activities such as arousal, thermoregulation, neuroendocine, and vegetative functions. H2-receptor antagonists are considered relatively benign, as observed with cimetidine, the primary adverse reaction is mental confusion. Cimetidine also inhibits hepatic oxidative metabolism by most cytochrome P450 enzymes; and, thus, inhibits the metabolism of a variety of drugs (including propranolol, carbamazepine, quinidine, theophylline, and certain tricyclic antidepressants). Other H2-receptor blockers (e.g., ranitidine, famotidine) do not seem to interfere with hepatic oxidation.

The H2 blockers are commonly used in treatment of peptic ulcer disease PUD (a disease in which ulceration occurs in the lower esophagus, stomach, duodenum, or jejunum). The most prominent symptom is gnawing pain that is relieved by food and alkali, but worsened by alcohol and condiments. The proximate cause of PUD is gastric acid hypersecretion.

The H2 blockers are conventionally used to treat peptic acid diseases include cimetidine, ranitidine, famotidine, and rizatidine. They are selective and do not block H1 receptors or have antimuscarinic activity. In addition, the blockade of central H2 receptors typically alters CNS neurotransmission; and may cause delirium, confusion, agitation, and seizures (rare). The pharmacokinetic properties of these compounds is summarized by Table 3 below.

TABLE 3
Pharmacokinetics*
Pharmacokinetic Properties of Histamine H2 Antagonists

| | | Time to | | | | | Elimination (%) | | |
|---|---|---|---|---|---|---|---|---|---|
| H2 | Bioavail- | Peak Plasma | Peak Plasma | Half- | | Volume of | Urine, | | |
| Receptor | ability | Concentration | Concentration | life | Protein | Distribution | Unchanged | | |
| Antagonist | (%) | (hrs) | 1 (mcg/ml) | (hrs) | Binding (%) | (L/kg) | Oral | IV | Metabolized |
| Cimetidine | 60-70 | 0.75-1.5 | 0.7-3.2 | ≈2² | 13-25 | 0.8-1.2 | 48 | 75 | 30-40 |
| | | | (300 mg dose) | | | | | | |
| | | | (3.5-7.5 IV) | | | | | | |
| Raniditine | 50-160 | 1-3 | 0.44-0.55 | 2-3³ | 15 | 1.2-1.9 | 30-35 | 68-79 | <10 |
| | (90-100 IM) | (0.25 IM) | (0.58 IM) | | | | | | |
| Famotidine | 40-45 | 1-3 | 0.076-0.1 | 2.5-3.5³ | 15-20 | 1.1-1.4 | 25-30 | 65-70 | 30-35 |
| | | | (40 mg dose) | | | | | | |
| Nizatidine | >90 | 0.5-3 | 0.7-1.8/1.4-3.6 | 1-2³ | ≈35 | 0.8-1.5 | 60 | na | <18 |
| | | | (150/300 | | | | | | |
| | | | mg dose) | | | | | | |

¹Dose dependent
²Increased in renal and hepatic impairment and in the elderly
³Increased in renal impairment
na = not applicable
*Reproduced from: Drug Facts and Comparisons, 1995, p. 1769.

Historically, the synthesis of H2 antagonists was achieved by stepwise modifications of the histamine molecule, which resulted in the first highly effective drug with potent H2-blocking activity, burimamide. This agent, like later compounds, retained the imidazole ring of histamine, but possessed a much bulkier side chain. Cimetidine, the first H2 blocker to be introduced for general clinical use, has won rapid acceptance for the treatment of ulcers and other gastric hypersecretory conditions; and has become one of the most widely prescribed of all drugs. This also led to the synthesis of numerous congeners.

Some pharmacokinetic information for these agents is discussed individually below:

Cimetidine: Absorption may be decreased by antacids, but is unaffected by food. Both oral and parenteral administration provide comparable serum levels. Plasma concentrations of 0.5 to 1 mcg/ml are required to suppress basal or gastric acid secretion; however, plasma concentrations of cimetidine have not correlated with duodenal ulcer healing. Blood concentrations remain above those required to provide 80% inhibition of basal, gastric acid secretion for 4 to 5 hours following a 300 mg dose. Cimetidine is widely distributed. Following oral administration, about 30% to 40% is metabolized in the liver, the sulfoxide being the major metabolite. Cimetidine is not significantly removed by hemodialysis or peritoneal dialysis.

Ranitidine: Absorption of oral ranitidine is not significantly impaired by the administration of food. Coadministration of antacids may reduce its absorption. Hepatic metabolism results in three metabolites. Maintenance of serum concentration necessary to inhibit 50% of stimulated gastric acid secretion (36 to 94 ng/ml) is 12 hours orally and 6 to 8 hours IV. Blood levels, however, bear no consistent relationship to dose or degree of acid inhibition.

Famotidine: Plasma levels after multiple doses of famotidine are similar to those after single doses. Famotidine is eliminated by renal (65% to 70%) and metabolic (30% to 35%) routes. The only metabolite identified is the S-oxide.

Nizatidine: A concentration of 1000 mcg/L is equivalent to 3 μmol/L; a dose of 300 mg is equivalent to 905 μmoles. Plasma concentrations 12 hours after administration are less than 10 mcg/L. Plasma clearance is 40 to 60 L/hour. Because of the short “half-life and rapid clearance, drug accumulation would not be expected in individuals with normal renal function who take either 300 mg at bedtime or 150 mg twice daily. Nizatidine exhibits dose proportionality over the recommended dose range.

### C. H3-Histamine Antagonists

H3 receptors are presynaptic regulators of synthesis and release of histamine into the synapse. Use of H3 receptors has been limited to experimental settings only. Accordingly, H3 histamine antagonists are not involved with and play no role in the present invention.

### II. A Preferred Protocol for Practicing the Invention

The treatment method comprising the present invention requires that at least one nonsedating H1-receptor histamine antagonist and at least one H2-receptor histamine antagonist be administered concurrently prior to imbibing alcohol. The preferred medicaments, mode of administration, and duration of mediating activity are described individually below.

Medicaments:

Among the presently available pharmaceutical choices for the nonsedating H1-antagonist are the piperidine derivatives fexofenadine, loratadine and desloratadine. Of these, the most preferred is loratadine (CLARITIN). For completeness, however, the preferred doses for all presently known nonsedating H1-antagonist deemed efficacious for use in the treatment method are given below by Table 4.

TABLE 4

| | Conventional Dosage | Preferred Single |
|---|---|---|
| Compound | Range (mg)^{@} | Dose (mg) |
| loratadine | 10-30 | 10 |
| desloratadine | 2-10 | 5 |
| fexofenadine | 50-200 | 100 |
| terfenadine | 60-100 | 60 |
| astemizole | 10-30 | 10 |

^{@}In accordance with the clinical experience of these drugs to date.

In the preferred method, the loratadine is in solid tablet form or is a gelatin capsule containing powdered loratadine; is a 10 mg concentrated dose; and is orally administered (by mouth; po) about 30-45 minutes prior to imbibing alcohol.

It is also envisioned and expected that, in addition to loratadine, desloratadine and fexofenadine, a diverse range of new variant compounds and formulations suitable as nonsedating H1-histamine antagonists will come into existence and be pharmaceutically available in the not too distant future. The primary characteristic of these new variants and formulations—which is shared in common with loratadine (CLARITIN) and also differentiates them from their predecessor 1^{st} generation H1 blockers—is that these new variant formulations do not cross the blood-rain barrier, or at least will do so minimally. This primary property and characteristic of not crossing the blood-brain barrier obviates those unwanted and undesired side-effects of antihistamines, such as cumulative drowsiness with alcohol, which would make the formulation inappropriate for use with controlling and avoiding alcohol-induced facial flushing.

Among the presently available pharmaceutical choices for the H2-antagonist are cimetidine, ranitidine, famotidine and nazatidine. Of these, ranitidine is most preferred. The desirable doses for each presently available agent to be administered are given by Table 5 below.

TABLE 5

| | Conventional Dosage | Preferred Single |
|---|---|---|
| Compound | Range (mg)^{@} | Dose (mg) |
| cimetidine | 100-400 | 300 |
| ranitidine | 100-200 | 150 |
| famotidine | 20-40 | 40 |
| nazatidine | 150-300 | 300 |

^{@}In accordance with the clinical experience of these drugs to date.

In the preferred method, the ranitidine is in tablet form; is a 150 mg concentrated dose; and is orally administered (by mouth; os) about 30-45 minutes prior to imbibing alcohol.

Doses and Dosages:

With regard to the dosages of the nonsedative H1- and H2-antihistamines employed in this treatment method, the doses disclosed by Tables 4 and 5 respectively and the frequency of their use are merely those presently endorsed by the manufacturers of each medicament and now approved by the FDA for medical use. However, it is believed and expected that effectively doubling the conventionally used therapeutic dose for each medicament employed will prove to be not only more efficacious in avoiding the flushing reaction, but also be substantially enhancing in the degree and effective duration of the treatment for the susceptible individual.

Alternative Medicament Formulations and Formats:

While the treatment methodology presently utilizes single doses of each medicament as solid tablets or gelatin capsules containing a powder, it is intended and expected that alternative formulations and formats of these medicaments will be manufactured and become available in the near future. Some illustrative instances of the envisioned new alternative medicaments are presented below.

(i) Some of the envisioned alternative formulations will be prepared and exist as extended (or timed) release capsules for each of the nonsedating H1-antagonists and H2-antagonists employed. These extended release formulations will bind the chosen medicament at increasing quantities and larger dosages as an active ingredient to a biodegradable matrix material for slow release within the body, a formulation and composition technique which is commonly used in pharmacology today to extend the uptake of any given drug within the body over time and to achieve a longer duration of desired pharmacological effect for the individual.

(ii) Among the envisioned alternative formats for delivering each medicament will be new preparations existing as lozenges, or applied via oral sprays, and/or introduced as liquid-centered sweets for each of the nonsedating H1-antagonists and H2-antagonists employed.

(iii) Still other envisioned alternative formats for delivering each medicament will appear as transcutaneous dermal patches and other transcutaneous delivery systems such as iontophoresis which are applied to the skin surface and rely upon passive and/or active mechanisms of action to deliver a concentrated dose of the chosen medicament across the skin.

Administration:

It is required for the treatment method that the chosen nonsedating H1 antagonist and the chosen H2 antagonist be administered concurrently. Accordingly, each pharmaceutical compound may be administered individually or be admixed prior to being administered. Also, the requirement of “concurrent” administration, by definition, includes and encompasses simultaneous, sequential and successive modes of administering each medicament.

It is also presently desirable, although not compulsory, that the route for administering each medicament be oral—i.e., by mouth. Given the present modes of formulation for each medicament as a solid powder or liquid preparation, it is expected that the individual will swallow the appropriate dose. Also, given the current pharmaceutical availability and packaging for each nonsedating H1-antagonist and H2-antagonist medicament, it is presently expected that each medicament will be a powdered solid which has been prepared into a tablet, caplet, or gelatin capsule form.

In the alternative circumstance, where it is either difficult or undesirable to administer each medicament by mouth, a parenteral mode of administration can and should be employed. By definition, a parenteral mode of administration is any manner of administration other than the oral route. Also, a parenteral route of administration should (and often will) be utilized in the future for each alternative medicament formulation and format (as described above) in order to accommodate and conform to the particular requirements of that alternative formulation or delivery format. Thus, for example, a transcutaneous dermal patch containing a concentrated dose of the chosen medicament must employ a transcutaneous mode of administration to be effective and to allow the medicament to pass across the skin into the dermis.

Duration of Treatment:

Based on anecdotal human case studies (which employed a single conventional dose of each required medicament), after the nonsedating H1 antagonist and the H2 antagonist have been concurrently administered, a lag period of about 30 minutes time usually occurs before the full effect of the orally administered medicaments is obtained. Then, after the initial 30 minute lag period has elapsed, the efficacy of the treatment method and the anti-flushing blockade for the individual remains in full effect for a time period ranging between 3½ and 4 hours in duration. It is believed also that a single dose treatment will counteract the flushing reaction caused by 4-6 ounces of alcohol (80-100 proof or 40-50% ethanol)—in any desired total beverage quantity. However, if the individual chooses to imbibe more than about 4-6 ounces of alcohol over a 3-4 hour period of time; or, alternatively, chooses to drink at the rate of about one ounce of alcohol per hour for more than 3-4 hours time at one drinking occasion; then the flushing blockade provided by the single dose treatment regimen will become ineffective as a consequence of the person continuing to drink alcohol.

As a precautionary measure also, the preferred mode of the treatment method does not presently expect nor presently allow for the taking of more than a single dose of the requisite medicaments over any 24 hour period of time. Clearly however, this precaution cannot and does not apply either to any alternative preparation such as the envisioned extended release formulations or to any alternative mode of administration expected in the future such as the use of transcutaneous dermal patches.

Nevertheless, since only single dose tablet or gelatin capsule preparations for each medicament are pharmaceutically manufactured and commercially sold today, the individual is therefore expressly warned against taking repeated or multiple doses of the presently available requisite medicaments seriatim, one after another. It is deemed unsuitable also for the individual to take any additional single dose tablets or capsules of these histamine antagonists after actually beginning to drink alcohol in any meaningful quantity.

### III. Anecdotal Human Studies

Case 1 Patient Treatment History:

Patient JC is a female, is 39 years old and weighs 123 lbs. She is taking no medication presently and also has no prior medical history of consequence.

Ms. JC drinks alcohol socially on occasion, usually in the form of wine. After imbibing several glasses of wine, however, JC consistently finds that she has a flushing of the face and torso. Via her own history and symptoms, Patient JC is demonstrably susceptible to alcohol-induced flushing.

Patient JC then underwent a protocol of medical treatment to control alcohol-induced flushing. In accordance with the treatment protocol, about thirty (30) minutes before the next social occasion when she was expecting (or intending) to imbibe wine or any other alcoholic beverage, JC orally self-administered 10 mg of loratadine [CLARITIN] in tablet form and 150 mg of ranitidine in tablet form concurrently. Subsequently, after then drinking 2-3 glasses of wine over several hours time, JC experienced no flushing reactions, either of the face or of the torso. Thus, the flushing blockade never became ineffective over the duration of the social drinking occasion. In addition, JC experienced no undesired side-effects whatsoever; in particular, JC felt no drowsiness or sedation over the entire duration of the flushing blockade and the social drinking occasion.

Case 2 Patient Treatment History:

Patient PH is a female, is 48 years old and weighs 132 lbs. She is taking no medication presently and also has no prior medical history of consequence.

Ms. PH drinks alcohol socially, usually in the form of mixed cocktails known as “cosmopolitans”, After imbibing several alcoholic cocktails, however, PH routinely finds that she has undergone a marked flushing of the face. Via her own history and symptoms, Patient PH is demonstrably susceptible to alcohol-induced flushing.

Patient PH then underwent a protocol of medical treatment to control alcohol-induced flushing. In accordance with the treatment protocol, about thirty (30) minutes before the next social occasion when she was expecting (or intending) to imbibe mixed cocktails or any other alcoholic beverage, PH orally self-administered 10 mg of loratadine [CLARITIN] in tablet form and 150 mg of ranitidine in tablet form concurrently. Subsequently, after then drinking cocktails over a time period of about 4 hours duration, PH experienced no flushing reactions of the face. Thus, the flushing blockade never became ineffective over the duration of the social drinking occasion. In addition, PH experienced no undesired side-effects whatsoever; in particular, PH felt no drowsiness or sedation over the entire duration of the flushing blockade and the social drinking occasion.

Case 3 Patient Treatment History:

Patient CG is a female, is 40 years old and weighs 115 lbs. She is taking Ambien (by prescription) presently; and also has a prior history of facial rosacea. She has no other medical history of consequence.

Ms. CG drinks alcohol socially on occasion, usually in the form of wine. By her own admission, CG typically drinks twenty (20) or more glasses of wine per week. After imbibing wine, however, CG consistently has headaches and has a marked flushing of the face and torso. Via her own history and symptoms, Patient CG is clearly susceptible to alcohol-induced flushing.

Patient CG then underwent a protocol of medical treatment to control alcohol-induced flushing. In accordance with the treatment protocol, about forty five (45) minutes before the next social occasion when she was expecting (or intending) to imbibe wine or any other alcoholic beverage, CG orally self-administered 10 mg of loratadine [CLARITIN] in tablet form and 150 mg of ranitidine] in tablet form concurrently. Subsequently, after then drinking multiple glasses of wine over four (4) hours time, CG experienced no flushing reactions of the face or of the torso. In addition, CG experienced no undesired side-effects whatsoever; in particular, CG felt no drowsiness or sedation over the entire duration of the flushing blockade and the social drinking occasion.

This protocol and regimen of advance medical treatment was then repeated on a second designated social/drinking occasion. For this 2nd episode, CG orally self-administered the same quantities of loratadine and cimetidine concurrently less than one hour in advance of ingesting wine. Subsequently, after drinking wine steadily for about four and one-half (4.5) hours, the flushing blockade remained effective.

On this 2nd occasion, however, CG continued her steady drinking of wine which then lasted for more than six (6) hours in all. CG became aware that the facial flushing blockage became ineffective—but only when continuing to drink after ingesting about 8 glasses of wine over 4 hours and 30 minutes time.

Case 4 Patient Treatment History:

Patient KK is a female, is 32 years old and weighs 128 lbs. She is allegeric to both aspirin and erythromycin; and is taking birth control medication. She has no other medical history of consequence.

Ms. KK drinks alcohol socially on occasion, usually in the form of beer and wine. After imbibing several glasses of beer or wine, however, KK routinely finds that a flushing of the face results. Via her own history and symptoms, Patient KK is susceptible to alcohol-induced facial flushing.

Patient KK then underwent a protocol of medical treatment to control alcohol-induced facial flushing. In accordance with the treatment protocol, about thirty (30) minutes before the next social occasion when she was expecting (or intending) to imbibe wine or any other alcoholic beverage, KK orally self-administered 10 mg of loratadine [CLARITIN] in tablet form and 150 mg of ranitidine in tablet form concurrently. Then subsequently, in spite of drinking 2 glasses of wine and 5 glasses of beer over more than six (6) hours drinking time, KK nevertheless experienced no facial flushing reaction. Thus, the flushing blockade never became ineffective over the more than six hour duration of KK's social drinking. In addition, KK experienced no undesired side-effects whatsoever; in particular, KK felt no drowsiness or sedation over the entire duration of the flushing blockade and the social drinking occasion.

Case 5 Patient Treatment History:

Patient KS is a female, is 26 years old and weighs 120 lbs. She is taking synthroid presently for the treatment of Grave's Disease. There is no other medical history of consequence.

Ms. KS drinks alcohol socially on occasion, with no particular preference as to alcoholic form. After imbibing several alcoholic drinks, however, KS consistently has a flushing of the face. Via her own history and symptoms, Patient KS is clearly susceptible to alcohol-induced facial flushing.

Patient KS then underwent a protocol of medical treatment to control alcohol-induced flushing. In accordance with the treatment protocol, about thirty (30) minutes before the next social occasion when she was expecting (or intending) to imbibe any alcoholic beverage, KS orally self-administered 10 mg of loratadine [CLARITIN] in tablet form and 150 mg of ranitidine in tablet form concurrently. Subsequently, after then imbibing about four alcoholic drinks over approximately three hours and forty five minutes drinking time, KS experienced no facial flushing reaction. Thus, the flushing blockade remained effective for the initial three hours and forty-five minutes duration of KS's social drinking. Moreover, KS experienced no undesired side-effects whatsoever; in particular, KS felt no drowsiness or sedation over the initial duration of the flushing blockade.

On this social occasion, however, KS decided to continue her drinking for several more hours time. Patient KS subsequently discovered—that after the initial three hours and forty-five minutes of effective flushing blockade had elapsed—she then experienced not only facial flushing, but also an increased heart rate and a hot sweating sensation over her torso as a consequence of her alcoholic intake.

Case 6 Patient Treatment History:

Patient OTT is a female, is 57 years old and weighs 124 lbs. She is presently taking non-steroidal anti-inflammatory compounds for headaches. There is no other medical history of consequence.

Ms. OTT drinks alcohol socially on occasion, but without preference as to alcoholic form. After imbibing several alcoholic drinks, however, OTT consistently finds that she has a flushing of the face and also experiences nasal congestion. Via her own history and symptoms, Patient OTT is demonstrably susceptible to alcohol-induced flushing.

Patient OTT then underwent a protocol of medical treatment to control alcohol-induced flushing. In accordance with the treatment protocol, about thirty (30) minutes before the next social occasion when she was expecting (or intending) to imbibe any alcoholic beverage, OTT orally self-administered 10 mg of loratadine [CLARITIN] in tablet form and 150 mg of rantidine in tablet form concurrently. Subsequently, after drinking 3 alcoholic beverages over about three hours and thirty minutes time, OTT experienced neither a facial flushing reaction nor any nasal congestion. Thus, the flushing blockade remained effective over the initial three hours and thirty minutes time of her social drinking occasion. In addition, OTT experienced no undesired side-effects whatsoever; in particular, OTT felt no drowsiness or sedation over the duration of the flushing blockade.

However, on this occasion, OTT chose to continue her consumption of alcoholic beverages beyond three drinks and for an extended period of time greater than three and one half hours. Unfortunately, the duration of effective facial flushing blockade did not extend beyond the initial three and one half hours time period; and OTT showed specific symptoms as a result of her continuing intake of alcohol, including facial flushing, an increased heart rate, and nasal congestion.

Case 7 Patient Treatment History:

Patient NS is a female, is 23 years old and weighs 118 lbs. She is not taking any medications presently; but suffers from eczema. There is no other medical history of consequence.

Ms. NS imbibes about 1-2 alcoholic drinks per week socially, and usually prefers either beer or spirits to other forms of alcohol. After imbibing several alcoholic drinks, however, NS typically notices that she has a marked flushing of the face. Via her own history and symptoms, Patient NS is susceptible to alcohol-induced facial flushing.

Patient NS then underwent a protocol of medical treatment to control alcohol-induced facial flushing. In accordance with the treatment protocol, about thirty (30) minutes before the next social occasion when she was expecting (or intending) to imbibe any alcoholic beverage, NS orally self-administered 10 mg of loratadine [CLARITIN] in tablet form and 150 mg of ranitidine in tablet form concurrently. Subsequently/after drinking 3 alcoholic beverages over about three to four hours time, NS experienced neither a facial flushing reaction nor any other unusual reaction to her intake of alcohol. Thus, the flushing blockade remained effective over the initial about three to four hours time of NS's social drinking. In addition, NS experienced no undesired side-effects whatsoever; in particular, NS felt no drowsiness or sedation over the duration of the flushing blockade.

However, on this occasion, NS decided to continue her consumption of alcoholic beverages beyond three drinks and for a period of time greater than four hours. Unfortunately, the duration of effective facial flushing blockade did not extend beyond the initial four hours time period for patient NS; and she subsequently showed both facial flushing and an increased heart rate as a result of her prolonged intake of alcohol.

Case 8 Patient Treatment History:

Patient TS is a male, is 62 years old and weighs 210 lbs. He is taking Lipitor (by prescription) and aspirin presently as medications; but has no prior medical history of consequence.

Mr. TS typically imbibes about 8 alcoholic drinks per week socially, but has no particular preference as to alcoholic beverage form. Also after imbibing alcohol, TS typically has a marked flushing of the face after only two drinks. Via his history and symptoms, Patient TS is remarkably susceptible to alcohol-induced facial flushing.

Patient TS then underwent a protocol of medical treatment to control alcohol-induced flushing; and the treatment efficacy was evaluated by three different occasions of social drinking thereafter. In accordance with the treatment protocol, about 30 minutes before each social occasion when he was expecting (or intending) to imbibe any alcoholic beverage, TS orally self-administered 10 mg of loratadine [CLARITIN] in tablet form and 150 mg of ranitidine in tablet form concurrently. Subsequently, on each of the first and second drinking episodes, TS imbibed three alcoholic drinks over three to four hours time. Notably, on neither of these initial two drinking episodes did TS experience any facial flushing reaction or any other unusual symptoms as a consequence of his drinking alcohol. Equally important, TS experienced no undesired side-effects whatsoever; in particular, TS felt no drowsiness or sedation over the duration of the flushing blockade.

This protocol and regimen of advance medical treatment was then repeated on a third designated social/drinking occasion; and TS again orally self-administered 10 mg of loratadine and 150 mg of ranitidine concurrently about 30 minutes in advance of ingesting alcohol. Subsequently, after imbibing at least four alcoholic drinks over about four hours time, the duration of the flushing blockade become ineffective. Patient TS then experienced the typical facial flushing reaction—but only at about four and one half hours time after he began to drink alcohol.

Case 9 Patient Treatment History:

Patient OCT is a female, is 54 years old and weighs 125 lbs. She is taking no medication presently and has no prior medical history of consequence.

Ms. OCT drinks alcohol socially on occasion, usually 1-2 alcoholic drinks; but has no preference as the form of alcohol. After imbibing alcohol, however, OCT recognizes that she has a flushing of the face. Via her own history and symptoms, Patient OCT is susceptible to alcohol-induced facial flushing.

Patient OCT then underwent a protocol of medical treatment to control alcohol-induced facial flushing. In accordance with the treatment protocol, about thirty (30) minutes before the next social occasion when she was expecting (or intending) to imbibe wine or any other alcoholic beverage, OCT orally self-administered 10 mg of loratadine [CLARITIN] in tablet form and 150 mg of ranitidine in tablet form concurrently. Subsequently, after then imbibing multiple alcoholic drinks over five (5) hours drinking time, OCT experienced no flushing reactions, either of the face or of the torso. Thus, the flushing blockade never became ineffective over OCT's five hour drinking occasion. Equally important, OCT experienced no undesired side-effects whatsoever; in particular, OCT felt no drowsiness or sedation over the duration of the flushing blockade.

Case 10 Patient Treatment History:

Patient ST is a female, is 21 years old and weighs 128 lbs. She is taking birth control medication presently, but has no other medical history of consequence.

Ms. ST typically enjoys 1-2 alcoholic drinks per week and has no preference as the form of alcohol. After imbibing alcohol, however ST recognizes that she has a marked flushing of the face. Via her own history and symptoms, Patient OCT is clearly susceptible to alcohol-induced facial flushing.

Patient ST then underwent a protocol of medical treatment to control alcohol-induced facial flushing. In accordance with the treatment protocol, about thirty (30) minutes before the next social occasion when she was expecting (or intending) to imbibe wine or any other alcoholic beverage, ST orally self-administered 10 mg of loratadine [CLARITIN] in tablet form and 150 mg of ranitidine in tablet form concurrently. Subsequently, after then imbibing several alcoholic drinks over five (5) hours drinking time, ST experienced no flushing reactions, either of the face or of the torso. Thus, the flushing blockade never became ineffective over ST's five hour social drinking occasion. Moreover, ST experienced no undesired side-effects whatsoever; in particular, ST felt no drowsiness or sedation over the duration of the flushing blockade.

The present invention is not to be restricted in scope nor limited in form except by the claims appended hereto.

1. A method for controlling alcohol-induced flushing in a susceptible human, said method comprising the steps of:
administering to the susceptible human an effective amount of at least one nonsedating H1-receptor histamine antagonist;
concurrently administering to the susceptible human an effective amount of at least one H2-receptor histamine antagonist; and
waiting a predetermined time period after said nonsedating H1-receptor histamine antagonist and said H2-receptor histamine antagonist are administered before the susceptible human imbibes alcohol.

2. The method as recited in claim 1 wherein alcohol-induced flushing of the face is controlled.

3. The method as recited in claim 1 wherein alcohol-induced flushing of the torso is controlled.

4. The method as recited in claim 1 wherein said nonsedating H1-receptor histamine antagonist and said H2-receptor histamine antagonist are administered together as a single prepared medicament.

5. The method as recited in claim 1 wherein said nonsedating H1-receptor histamine antagonist and said H2-receptor histamine antagonist are administered separately as individually prepared medicaments.

6. The method as recited in claim 1 wherein said nonsedating H1-receptor histamine antagonist is a peripherally selective agent.

7. The method as recited in claim 1 wherein said nonsedating H1-receptor histamine antagonist is a piperidine derivative.

8. The method as recited in claim 1 wherein said nonsedating H1-receptor histamine antagonist is selected from the group consisting of terfenadine, astemizole, and fenoxfenadine.

9. The method as recited in claim 1 wherein said nonsedating H1-receptor histamine antagonist is loratadine.

10. The method as recited in claim 1 wherein said H2-receptor histamine antagonist is selected from the group consisting of cimetidine, famotidine and nizatidine.

11. The method as recited in claim 1 wherein said H2-receptor histamine antagonist is ranitidine.

12. The method as recited in claim 1 wherein said nonsedating H1-receptor histamine antagonist and said H2-receptor histamine antagonist are administered orally to the susceptible human.

13. The method as recited in claim 1 wherein said nonsedating H1-receptor histamine antagonist and said H2-receptor histamine antagonist are administered parenterally to the susceptible human.
